(19) United States
(12) Patent Application Publication
(10) Pub. No.: US 2019/0187154 A1
(43) Pub. Date: Jun. 20, 2019

(54) BIOMEDICAL MEASURING DEVICES, SYSTEMS, AND METHODS FOR MEASURING PEPTIDE CONCENTRATION TO MONITOR A CONDITION

(71) Applicant: Jana Care, Inc., Boston, MA (US)

(72) Inventors: Ashok A. Kumar, Boston, MA (US); Jeremy E. Schonhorn, Boston, MA (US); Daniel B. Lookadoo, Boston, MA (US); Abha Patil, Boston, MA (US); Lara Murray, Boston, MA (US); Harshit Harpaldas, Boston, MA (US); Sidhant Jena, Boston, MA (US); Michal Depa, Boston, MA (US)

(21) Appl. No.: 16/222,463

(22) Filed: Dec. 17, 2018

Related U.S. Application Data
(60) Provisional application No. 62/599,553, filed on Dec. 15, 2017.

(51) Int. Cl. G01N 33/68; G01N 33/92; G01N 33/74; G01N 33/49; G01N 21/17; G01N 33/538

(52) U.S. Cl. CPC G01N 33/6854; G01N 33/92; G01N 33/74; G01N 33/48771; G01N 21/17; G01N 33/538; G01N 33/6893; G01N 33/49

(57) ABSTRACT

Systems and methods directed to a monoclonal antibody covalently conjugated to latex in a two-step process to be used with a test strip and mobile-phone connected fluorimeter device. The test strip is combined with a method of analysis for quantitative detection of NT pro-BNP using the mobile device. A method for NT-proBNP testing system includes reading the test at an initial time point and at specific intervals during development of the test result, the dynamic behavior of the test can be used to distinguish differences between samples that would otherwise be difficult to differentiate by an end-point measurement due to the hook effect. Using two fluorescent tags with different excitation wavelength or emission wavelengths, or two colored beads with different absorption wavelengths, the test and the control line can simultaneously be read as they develop and dynamic formation can be used to distinguish high levels.

### RELATED APPLICATION

The present application claims the benefit of U.S. Provisional Application No. 62/599,553 filed Dec. 15, 2017, which is hereby incorporated herein in its entirety by reference.

### TECHNICAL FIELD

Embodiments related generally to immunoassays for monitoring a condition of a user, such as congestive heart failure, and more specifically to methods and systems to measure a level of N-terminal pro B-type Natriuretic Peptide in a blood sample using a test strip and a portable optoelectronic reader paired to a mobile device.

### BACKGROUND

An immunoassay is a procedure for detecting or measuring specific proteins or other substances through their properties as antigens or antibodies. Recent developments in immunoassays include point-of-care tests which utilizes an optoelectronic device or accessory for performing immunoassays, such as, but not limited to, fluorescent immunochromatographic assays, in combination with a mobile device, such as a smart phone, laptop, tablet, bracelet, smart watch, or other portable device, to process the data collected from the optoelectronic device and monitor a condition of a user.

One such condition is heart failure. Heart failure is responsible for 11 million physician visits each year, and more hospitalizations than all forms of cancer combined. Of those 11 million physician visits, congestive heart failure (CHF) is the first-listed diagnosis in over 875,000 hospitalizations and it is the most common diagnosis in hospital patients age 65 years and older. With nearly 550,000 new cases are diagnosed in the U.S. each year, the direct costs associate with CHF are estimated as high as $38 billion annually. The crisis of heart disease and CHF will continue to grow as the population ages, thus the discernment of new diagnostic strategies to improve prognosis and reduce costs is critical.

Current therapies for CHF include the use of angiotensin converting enzyme (ACE) inhibitors and beta-blockers. However, these agents are under-utilized and may be dosed inadequately when employed.

Markers of cardiac neurohormonal activation, particularly N-terminal pro-B-type natriuretic peptide (NT-proBNP), have been identified as possible tools to identify and treat patients with CHF. B-type (or brain) natriuretic peptides affect fluid homeostasis (through natriuresis and dieresis) and vascular tone (through decreased angiotensin II, norepinephrine synthesis), both essential components in the pathophysiology of CHF. In humans, NT-proBNP is found in largest concentration in the left ventricular (LV) myocardium, but are also detectable in atrial tissue as well as in the myocardium of the right ventricle.

A significant body of evidence has been developed to demonstrate that NT-proBNP levels correlate with diagnosis, clinical status, and prognosis in CHF, and may be useful for the longitudinal management of patients with CHF. NT-proBNP levels are highly sensitive and specific for the diagnosis of acute CHF and are correlated with the severity of CHF symptoms. Studies have shown that NT-proBNP levels were significantly higher in patients with decompensated CHF and that as CHF was treated, NT-proBNP levels fell in tandem. Consequently, an elevated NT-proBNP has proven to be one of the single strongest independent predictors for the final diagnosis of acute CHF and monitoring of NT-proBNP levels adds significantly more diagnostic power to the clinical history of a patient with heart issues.

During the past decades, several analytical methods have been developed for the detection of serum levels of NT-proBNP, including radioimmunoassay (MA), immunoradiometric assay (IRMA), the enzyme-linked immunosorbent assay (ELISA), and recently-developed electrochemiluminescence immunoassay (ECLIA). However, ELISA requires a higher sample volume and a long detection time, MA and IRMA develop radionuclide pollution problems and less automation, and although ECLIA is characterized by high sensitivity, specificity, and easily-operated automation, it still needs high cost, laboratory-oriented large analytic instrument, well-trained personnel sample transportation and requires increased waiting time.

Recent developments for the detection of NT-proBNP include a POCT and the use of a fluorescent immunochromatographic assay. Based on a sandwich-type immunoassay format, analytes in samples were captured by one monoclonal antibody labeled with fluorescent protein and “sandwiched” by another monoclonal antibody immobilized on a nitrocellulose membrane. The fluorescence and concentration of analytes were measured and calculated by fluoroanalyzer. This method of NT-proBNP detection applies a fluorescent protein modified by streptavidin as a tracer to conjugate the biotin-labeled anti-NT-proBNP antibody on the conjugation pad to facilitate the detection of NT-proBNP in human serum samples. This type of testing provides rapid and accurate testing results for patients, and improves therapeutic strategies, but current POCT for NT-proBNP are either not simple enough to be performed outside of a certified clinical laboratory or not cost effective for use in point-of-care settings. To render the NT-proBNP test more affordable and easy to be used by medical professional or by a patient at home, a POCT using a simple assay and device is desired.

Among current fluorescence detection devices, a digital camera component or high speed electronic component is most commonly used to detect fluorescent proteins. U.S. Patent Application Publ. No. 2015-0056719 A1 to Karlovac requires the use of a digital camera as a core component of a universal rapid diagnostics test reader; U.S. Pat. No. 7,416,700 to Buechler uses an assay mechanism requiring drive electronics to position an assay with respect to an optical energy source and optical energy detector; Canadian Patent No. 2,132,707 to Studholme requires a laser diode for exciting the sample to be assayed. Other fluorescence methods use different means and methods to detect fluorescence that does not lend itself to point-of-care testing. U.S. Pat. No. 5,994,707 to Mendoza requires a fluorescence spectroscopy method using an optical interface that is exclusively fiber optic based, and U.S. Pat. No. 8,824,800 to Bremnes requires a chromatographic test strip as well as an image of the test strip to complete an assay. The components of these detection devices limit the minimum cost and size of a detection system.

To measure NT-proBNP, various methods have been disclosed. U.S. Pat. No. 7,507,550 to Spinke discloses an analytical sandwich test for determining NT-proBNP comprising at least two antibodies to NT-proBNP, wherein at least one of the antibodies to NT-proBNP is a monoclonal antibody. One of these antibodies is directed at least against parts of the epitope of NT-proBNP comprising the amino acids 38 to 50. In addition, one of these antibodies is directed at least against parts of the epitope of NT-proBNP comprising the amino acids 1 to 37 or 43 to 76. The epitope recognized by the antibodies can also slightly overlap.

U.S. Patent Application Publ. No. 2009-0163415 A1 to Katrukha discloses antibodies against glycosylated forms of proBNP and NT-proBNP, which may be utilized as an antigen for antibody generation as well as a calibrator or immunological standard in different types of immunoassays. This disclosure also relates to a stable standard or calibrator pro-Brain Natriuretic Peptide (proBNP) preparation for use in a method for detecting BNP immunoreactivity in a sample, the preparation comprising glycosylated proBNP or a fragment thereof. In addition, the disclosure is directed to an assay for precisely detecting the NT-proBNP circulating in a patient's blood, wherein the level of glycosylation of the proBNP molecule is exploited.

U.S. Pat. No. 7,175,992 to Fong discloses methods for quantitatively measuring the amount of an analyte of interest in a fluid sample, and kits useful in the methods. The methods involve providing a solid phase apparatus comprising a membrane having an application point, a sample capture zone, and a control capture zone, where the sample capture region is between the contact region and the control capture zone; and providing a sample collection apparatus comprising a population of analyte binding particles or a population of analyte coated particles. In the assays, a fluid sample is introduced into the sample collection apparatus, and the resultant mixture is applied to the application point of the membrane. The fluid allows transport components of the assay by capillary action to and through the sample capture zone and subsequently to and through the control capture zone. The amount of analyte in the fluid sample is related (e.g., either directly or inversely) to a corrected particle amount, which can be determined, for example, as a ratio of the amount of particles in the sample capture zone and the amount of particles in the control capture zone.

U.S. Pat. No. 6,509,196 to Brooks discloses a compensation for non-specific signals in quantitative immunoassays. The method disclosed involves providing a membrane having an application point, a detection zone, and a contact region, where the contact region is between the application point and the detection zone, and has test particles and internal control particles imbedded within it; contacting the application point with the fluid sample; maintaining the membrane under conditions sufficient to allow fluid to transport analyte by capillary action to the contact region, where the analyte binds to the test particles; further maintaining the membrane under conditions sufficient to allow fluid to transport analyte-bound test particles and internal control particles to the detection zone, where they interact with a detection reagent; and detecting the amount of test particles and the amount of internal control particles in the detection zone. The amount of analyte in the fluid sample is related to a corrected test particle amount, which can be determined, for example, as the difference between the amount of test particles and the amount of internal control particles in the detection zone. Alternatively, the fluid sample can be contacted with the detection zone of the apparatus, and the test particles are mobilized by addition of fluid to the application point. Other methods involve providing a solid phase reactant for an enzyme-linked immunosorbent assay, and contacting the solid phase reactant with a solution containing an initial detection antibody and an internal control antibody. The amount of analyte in the fluid sample is related to a corrected amount of initial detection antibody, which can be determined, for example, as the difference between the amount of initial detection antibody and the amount of internal control antibody on the solid-phase reactant. Another method for quantitatively evaluating lateral flow immunoassays is described in the publication by Rey, E. G., O'Dell, D., Mehta, S. & Erickson, D., entitled “Mitigating the Hook Effect in Lateral Flow Sandwich Immunoassays Using Real-Time Reaction Kinetics;” Anal. Chem. 89, 5095-5100 (2017). This publication describes a method to do time based readings of both test and control line to use the ratio as a parameter to evaluate the test and overcome the hook (or prozone) effect.

Despite the prior art, examples of quantitative lateral flow test systems remain limited in practice. The reliance on image-based analysis for fluorescent lateral flow readers has limited the development of robust, portable, and inexpensive fluorescent readers. In the particular case of NT-proBNP, the wide natural range of the analyte—from single digits to tens of thousands of pg/mL—introduces challenges to achieve sufficient precision and accuracy across the clinically relevant range. Thus, there still remains a need for a widely available, cost effective, and accurate POCT diagnostic tool for the detection of NT-proBNP to identify and manage those with CHF.

### SUMMARY

Embodiments of the present invention are directed to systems and methods for detecting a concentration of a protein or peptide (oligo- or poly-) or other marker or analyte in a carrier such as blood, plasma, serum, saliva, sweat, urine, or any suitable carrier to monitor a condition, the system including, for example, (i) a test strip specifically configured to isolate the marker for optoelectronic reading, (ii) an optoelectronic reader, (iii) methods for analyzing signals, and optionally (iv) a test strip holder. For sake of efficiency, embodiments described herein are specific to systems and methods for detecting the concentration of NT-proBNP in blood, serum, or plasma for monitoring a heart conditions such as heart failure. However, one or ordinary skill in the art would recognize that the systems and methods can be configured to detect a concentration of another protein, or disease marker, for monitoring conditions other than heart failure.

In embodiments, a system for detecting the concentration of NT-proBNP in blood, serum, or plasma comprises (i) a test strip, (ii) an optoelectronic reader, (iii) methods for analyzing signals, and optionally (iv) a test strip holder. The test strip can be a type of lateral flow immunoassay with a fluorescent signal. The optoelectronic reader is preferably a compact, handheld device that operably connects to a mobile phone or tablet device and is capable of both exciting the fluorescence of the test strip and reading the emitted signal. The methods for analyzing signals include ways to compensate for sample and test variability, to overcome the high-dose hook effect, and to detect errors in the test procedure.

In one embodiment, a mobile device is used to measure various biological and chemical analytes in either a liquid or solid substrate with the use of a mobile-phone connected fluorimeter that provides quantitative readings for fluorescence-based analytical tests. One or more light sources, at least one of which emits UV light, are combined with an optical path component that minimally absorbs visible and UV light. A photodetector with organic pigment filters for color selection or a photodetector with Gaussian filters are integrated onto a chip with an optional filter gel or glass filter element. The completely assembled fluorescence detection device, summarily referred to as the optoelectronic reader, connects to a mobile device such as a mobile phone though a headphone jack, Bluetooth, or other connection. This mobile device enables analysis and parameters to be set, as well as data to be logged and transmitted.

In an embodiment, a monoclonal antibody is covalently conjugated to latex in a two-step process to be used with a test strip. The test strip is combined with a method of analysis for quantitative detection of NT-proBNP using the optoelectronic reader and the mobile device.

In an embodiment, a method for NT-proBNP testing system includes reading the test at an initial time point and at specific intervals during development of the test result, the dynamic behavior of the test can be used to distinguish differences between samples that would otherwise be difficult to differentiate by an end-point measurement due to the hook effect. The test strip consists of a test line that captures the target antigen and, in one embodiment, includes a control line that captures a reference material. Using a combination of reflectance photometry and fluorescence, of two fluorescent tags with different excitation wavelength or emission wavelengths, or of two colored beads with different absorption wavelengths, the test, and the control line can simultaneously be read as they develop and dynamic formation can be used to distinguish high analyte concentrations. In cases where a fluorescent tag is used for one measurement with a visible tag for the other measurement, a low signal interference between the measurements is enabled when reading the test.

Quantitative readers for fluorescent immunoassays generally require a full 2-dimensional image for analysis whereas the embodiment described here enables the use of a detector that aggregates photometric data into a single point. The use of photodiodes and other such point detectors enables a more compact and affordable device. The restriction to image-based analysis is generally regarded as a necessary means to compensate for background signals and ensure that the assay has run successfully by measuring a distinct control line. The embodiments described herein provide combinations of techniques that enable these restrictions to be overcome by the use of a fluorescent reference line, co-locating control lines, and using different wavelengths to spectrally separate information that is spatially co-located.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1A is a perspective view of a test strip according to an embodiment of the invention.

FIG. 1B is an exploded view of a test strip assembly according to an embodiment of the invention.

FIG. 2A is a cross-sectional view of a wrap around strip assembly of FIG. 1A, according to an embodiment of the invention.

FIG. 2B is a cross-sectional view of a reverse sample pad with extended tongue assembly of FIG. 1A according to an embodiment of the invention.

FIG. 2C is a cross-sectional view of a UV transparent backing assembly of FIG. 1A, according to an embodiment of the invention.

FIG. 3 is a cross-sectional view of a lateral flow immunoassay design common in prior art.

FIG. 4A is a perspective view of the top part of an embodiment of a test strip holder according to an embodiment of the invention.

FIG. 4B is a perspective view of the bottom part of an embodiment of a test strip holder according to an embodiment of the invention.

FIG. 4C is a perspective view of an assembled embodiment of a test strip holder according to an embodiment of the invention.

FIG. 4D is a cross-sectional view of an assembled embodiment of a test strip holder according to an embodiment of the invention.

FIG. 5A is a cross-sectional view of a first schematic of optical components of an analyzer component with an optional optical filter according to an embodiment of the invention.

FIG. 5B is a cross-sectional view of a second schematic of optical components of an analyzer component with an optional optical filter according to another embodiment of the present invention.

FIG. 6 is a graph comparing the calculated values of NT-proBNP from the present invention to values of spike material references to a large clinical analyzer (i.e., the Roche Elecsys system).

FIG. 7 is a graph of the signal of the present invention measured over the course of time after correction according to an embodiment of the present invention.

FIG. 8 is a schematic of primary components of the optoelectronic reader described according to an embodiment of the present invention.

FIG. 9 is a schematic of the processes performed by the optoelectronic reader and mobile device over the course of a test according to an embodiment of the present invention.

FIG. 10 is a schematic according to an embodiment of the present invention wherein a reference line is used to normalize the test signal.

FIG. 11 is a schematic according to an embodiment of the present invention wherein a co-printed control line is used to normalize the test signal.

FIG. 12 is a schematic according to an embodiment of the present invention of the flow of information of the test system integrated into a health care program.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

The embodiments described below are not intended to be exhaustive or to limit the invention to the precise forms disclosed in the following detailed description. Rather the embodiments are chosen and described so that others skilled in the art may appreciate and understand the entire disclosure.

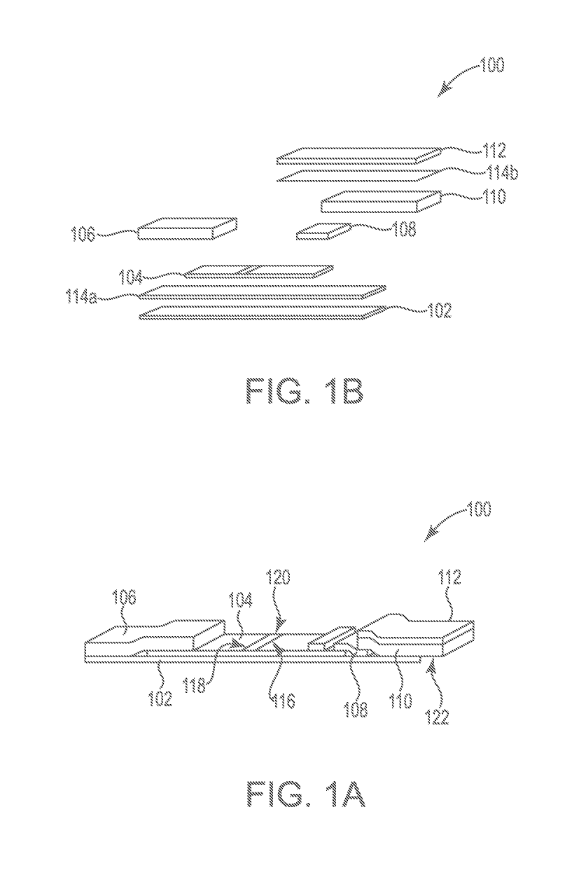

Referring to FIGS. 1A and 1B, a biomedical measuring device comprises a composite test strip assembly 100 used for applying a sample and for inserting such sample laden strip into an optical sensing and reading apparatus for analysis of the sample. In the embodiment depicted, test strip assembly 100 comprises eight components. In alternative embodiments, more or less than eight components may be utilized.

Test strip assembly 100 can comprise a solid backing material layer 102, a hydrophilic wicking membrane 104 coupled on a first surface to backing material layer 102 via adhesive layer 114a. Backing material 102 can comprise a support layer formed of a polymeric or plastic film material that can be of any color or be optically clear. Backing material does not absorb water. Backing material layer 102 can be formed of, for example, polyester, polycarbonate, vinyl, or combinations thereof. In particular embodiments, a desired thickness of backing material layer 102 is in a range of from about 0.1 to about 10 mm. Hydrophilic wicking membrane 104 can be formed from nitrocellulose (backed or unbacked) and is sensitized to NT-proBNP through a test line 116 and to a control material, such as horseradish peroxidase, through a control line 118.

An absorbent end pad 106 is coupled to one end of hydrophilic wicking membrane 104, with an overlap of from about 1 to about 5 mm, on a surface opposite to which backing material layer 102 is coupled. Absorbent end pad 106 is formed of a material that provides sufficient wicking capacity to pull 130-200 μL of fluid or more over the strip in 20 minutes or less. End pad 106 can comprise one-direction or multi-direction woven fiber, or alternatively a non-woven material such as a spun-bonded or plexifilamentary absorbent material. The fiber material can comprise, for example, nylon, fiberglass, a superabsorbent polymer such as a hydrogel, cellulose, or combinations thereof. In particular embodiments, a desired thickness of pad 106 is in a range of from about 0.1 to about 1 mm.

A conjugate absorbent pad 108 is coupled to the other end of hydrophilic wicking membrane 104 on the side opposite to which backing material layer 102 is coupled. Conjugate absorbent pad 108 contains reporter particles sensitized to either the test material or the control material and overlaps wicking membrane 104. Conjugate absorbent pad 108 is then overlapped by a sample pad 110 which serves to introduce samples into the test strip assembly 100, while simultaneously retaining red blood cells.

A top layer, cover film 112 backed with adhesive layer 114b, is coupled to sample pad 110 and provides coverage of sample pad 110, conjugate absorbent pad 7, and part of membrane 104, while leaving test line 116 exposed. Top film layer 112 can be formed from a plastic or polymeric material that exhibits a balance between a moderate flexural modulus (e.g. from about 100,000 to about 600,000 psi), and good tensile strength (e.g. from about 3000 to about 15000 psi). This allows for ease in manufacturing, yet is still rigid enough for performing the assay. Suitable materials include, for example, acetal copolymer, acrylic, nylon, polyester, polypropylene, polyphenylene sulfide, polyetheretherketone, poly(vinyl chloride), or combinations thereof.

In this particular embodiment, the assembled strip 100 as depicted in FIG. 1B is designed such that a sample addition area 122 is on the opposite side as a sample signal area 120.

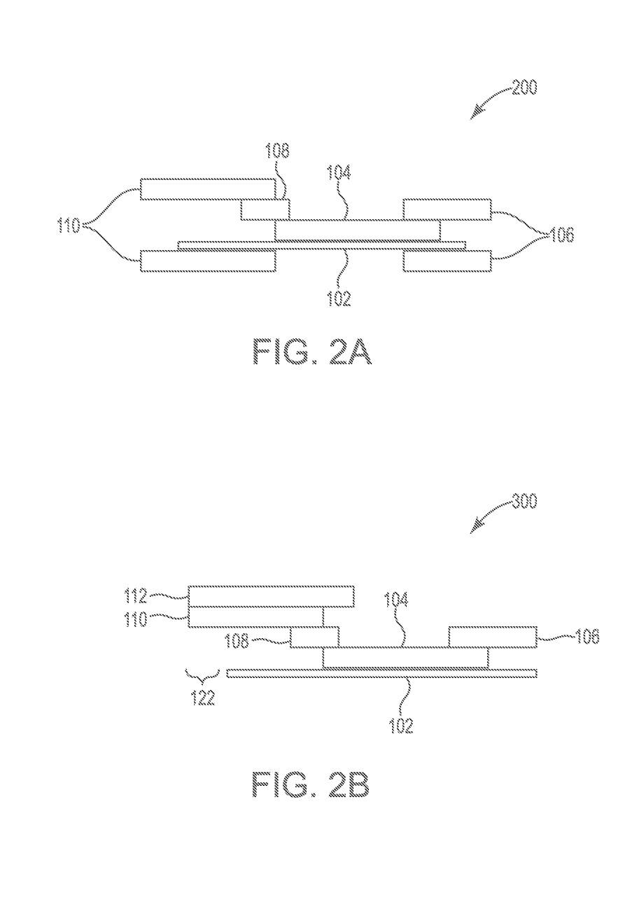

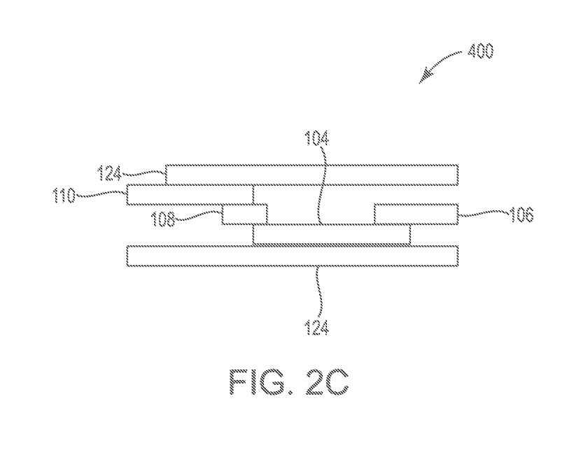

Referring now to FIGS. 2A-2C, cross-sectional views are depicted of multiple embodiments of strips that meet the functional requirements of the strip in FIGS. 1A and 1B. All of the described strip embodiments are designed such that a liquid sample can be applied on the opposite face of the strip as the side where the signal is detected.

Referring to the alternative embodiment depicted in FIG. 2A, referred to as a wrap around strip assembly 200, sample pads 110 and absorbent end pads or wicks 106 are folded around the ends of a vinyl backing layer 102. In this embodiment, a sample is added to the sample pad 110 on the side opposite of a nitrocellulose wicking membrane 104. In this embodiment, the sample fluid wicks around sample pad 110, mobilizes the conjugate, and flows across wicking membrane 104. The extension of the wick or end pad 106 around backing layer 102, increases the wicking capacity while keeping a short length between the end of the strip 100 and the test line 116.

Referring now to FIG. 2B, referred to as a reverse sample pad with extended tongue assembly 300, sample pad 110 overhangs the backing layer 102 such that the sample can be added to sample pad 110 at 122 opposite of wicking membrane 104.

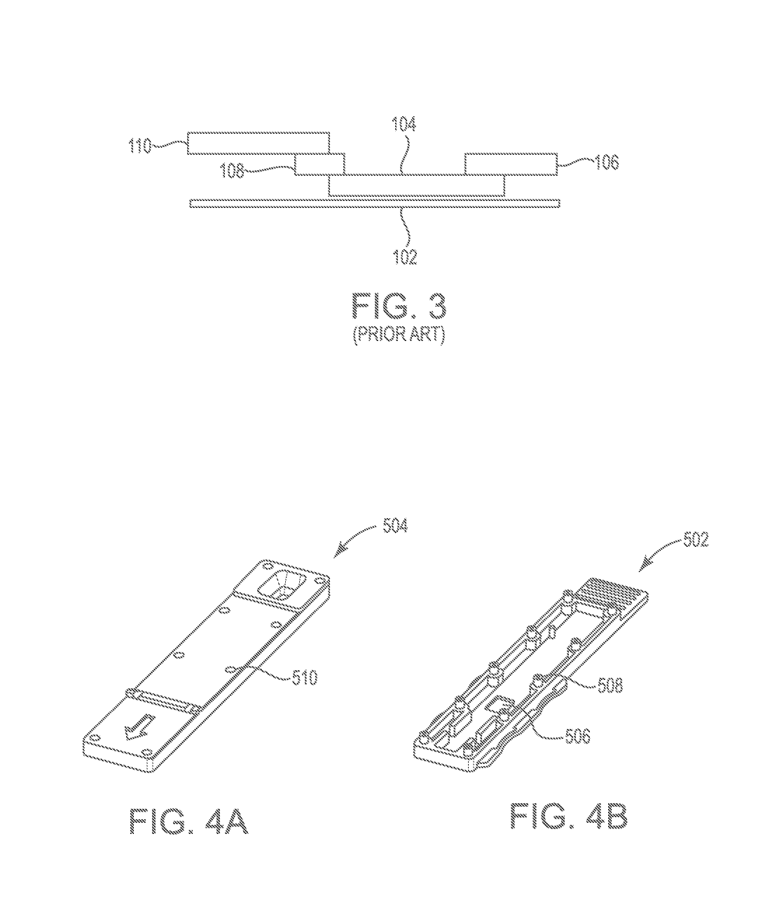

Referring now to FIG. 2C, referred to as a UV transparent backing assembly 400, strip assembly 400 is assembled similarly to the typical lateral flow assay 10 of FIG. 3, with the significant exception that instead of an opaque backing B of FIG. 3, backing layer 124 is optically clear in the 350-800 nm range, thus allowing UV light and emitted red light to pass through. As depicted in FIG. 3, prior art on lateral flow immunoassays primarily teaches designs 10 wherein a sample is applied on the same face as where the signal is developed and read. In FIG. 3, backing layer B is made of an opaque plastic material that does not absorb water and is coated with an adhesive layer. A wicking membrane M, such as nitrocellulose wicking membrane 104, is applied to the backing layer B and then overlapped on one end by an end pad P, similar to end pad 106, and on the other end by a conjugate pad C, similar to conjugate pad 108, which is further overlapped by a sample pad S, similar to sample pad 110.

By designing the test strip in such a way as to have these two actions (i.e. sample addition and test reading) on opposite faces, various embodiments may enable versatility in how the test can be read. This design allows a compact optoelectronic reader, as described as part of this disclosure, to be used for measuring the test strip.

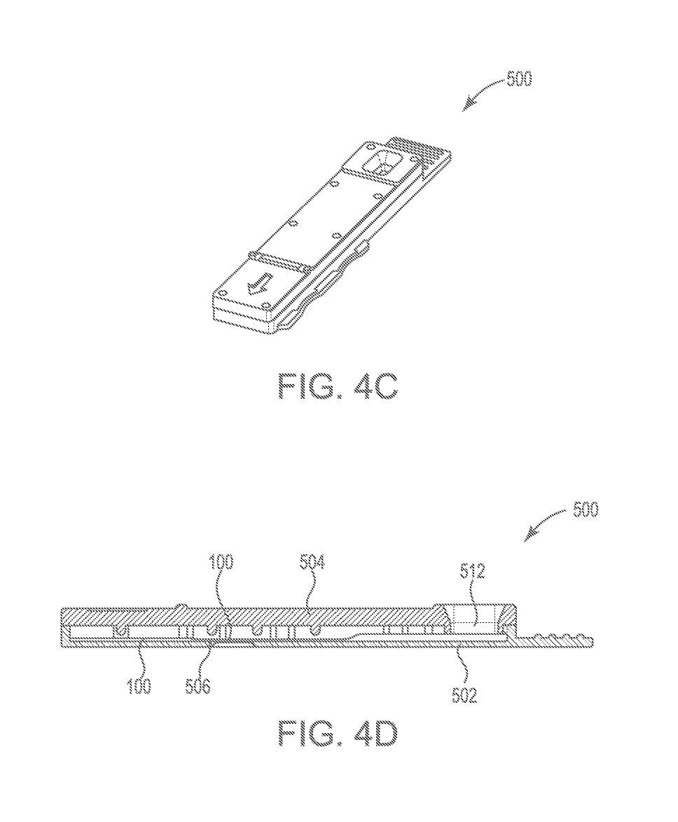

Referring now to FIG. 4A-4D, a test strip holder 500 is depicted for the test strip depicted in FIG. 1. The test strip is placed in cassette bottom 502 with the test line area 120 facing a window 506 of bottom 502 of holder 500. Cassette top 504 is then placed on top of cassette bottom 502 and fitted by mating pegs 508 of bottom 502 with apertures 510 of top 504. Features in bottom 502 can be present to adjust the positioning of the test line over window 506, and can also be present to hold the test strip firmly in position. Feature 512 provides a sample port in which to add sample to the sample pad 110.

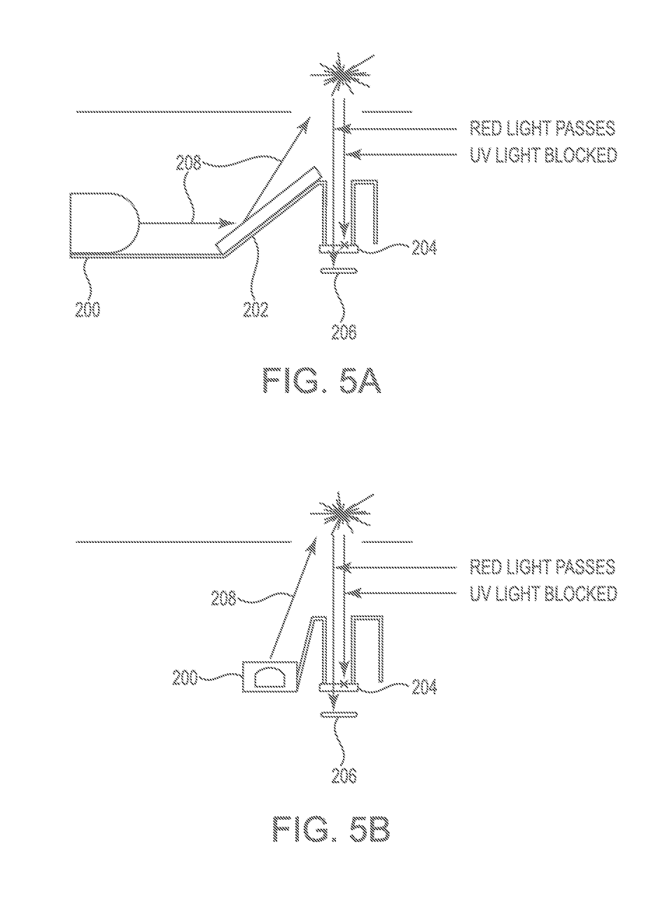

Referring now to FIG. 5A-5B, an embodiment of an optoelectronic reader system 600 for the test strip is depicted. System 600 may be configured in multiple ways. In one embodiment, FIG. 5A, an ultraviolet light source 200, such as an LED, shines on a reflective surface 202 that redirects the light path 208 to the test zone of the lateral flow test strip. The ultraviolet light excites the fluorescent particles and emitted light passes down to an image-based reader, such as a photodetector 206. Optionally a filter element 204 can be placed in the light path to remove stray ultraviolet light.

An alternative embodiment is depicted in FIG. 5B, where the light source 200 is a surface mounted LED that is positioned near to the photodetector 206 such that a significant portion of the emitted light 208 shines directly on the test zone of the lateral flow test strip. The ultraviolet light excites the fluorescent particles and emitted light passes down to a photodetector 206. Optionally a filter element 204 can be placed in the light path to remove stray ultraviolet light. Referring now to an exemplary, non-limiting design of the immunoassay of the test strip, the assay uses a monoclonal antibody, 15F11 (a.a.r 13-24) bound to the solid phase, and monoclonal antibody 24E11 (a.a.r. 67-76) conjugated to from about 0.1 to about 0.4 micron microparticles or beads, and more particularly, in one non-limiting embodiment, 0.3 micron Europium chelate carboxylated polystyrene microparticles (latex). A non-limiting alternative design of the test strip uses monoclonal antibody 16E6 (a.a.r.) in place of monoclonal antibody 24E11. Monoclonal antibody 24E11 (24E11) is covalently conjugated to latex in a two-step process. In the first step, latex is primed by treating with 1-ethyl-3-(-3-dimethylaminopropyl) carbodiimide hydrochloride (EDC) and N-hydroxysulfosuccinimide (NHS). In one embodiment, the amount of EDC is between 0.1 and 2 mg/mg of latex with 0.2 mg EDC/mg of latex. The amount of NHS used is between 5-fold and 20-fold molar excess to EDC. In one non-limiting embodiment, the amount of NHS is a ten-fold molar excess to EDC. Latex priming may be performed over broad pH range of 5 to 9. In one non-limiting embodiment, the priming pH is 6.5. The 24E11 latex conjugate is prepared by adding the antibody to the primed latex where the latex to antibody ratio is between 5:1 and 100:1. Successful conjugates can be prepared over a broad pH range from pH 5-9, but i one non-limiting embodiment, the coupling pH range is from 5.5 to 6.5 in MES buffer.

In alternative embodiments to the EDC NHS chemistry described above, DCC may be substituted for EDC, although it is not readily water soluble. Other conjugation methods include, but are not limited to: passive coupling, a method incorporating biotinylation of antibody and binding to avidin, neutravidin, and/or streptavidin particles; amine modified/hydrazide modified beads conjugation to periodate-oxidized antibodies; amine modified beads activated with glutaraldehyde linked to antibodies containing available amine groups; and chloromethyl-modified beads linked to amine groups on antibodies.

Referring back to FIG. 1A, control line 118 and test line 116 are offset physically. In an alternative embodiment control line 118 and test line 116 are in the same physical position. For the control line, any non-interfering antibody and conjugate system can be used. Non-limiting examples include chicken IgY/monoclonal or polyclonal anti-chicken IgY antibodies and horseradish peroxidase/monoclonal or polyclonal anti horseradish peroxidase antibodies. The reporter antibody for the control line can be linked to either colloidal gold, colored latex, or fluorescent beads. By choosing a reporter that does not overlap with the signal of the test line (by having a different excitation wavelength, emission wavelength, or absorption wavelength) a single region can be multiplexed with the optoelectronic reader described above to read multiple analytes at once. Alternatively, in some embodiments, control line 118 may be omitted entirely.

It is further contemplated that additional test lines for other analytes can be co-printed to enable further multiplexing of the test. By using differences in either the excitation or emission wavelength for fluorescent tags, or of different absorbance peaks for tags measured by reflectance, many analytes can be read at the same test zone.

In one embodiment, the test line solution may also be mixed with a fluorescent reference material, such as fluorescein, Eu-chelate, or similar materials. The material may either be deposited in solution as solubilized material or on a supporting matrix, such as a polymer bead. This reference line is measured when the strip is inserted as a way to account for the path length between the detector and the sample as well as the intensity of the UV light. Upon the addition of sample and wash buffer, the signal from the material is quenched or the material is washed away, thus eliminating interference from the reference material and the test material.

### Test Strip Design

One method for sensitizing the nitrocellulose includes the following steps. Monoclonal antibody is deposited onto backed or unbacked nitrocellulose membrane using equipment that can deliver dots or lines. In one embodiment, the nitrocellulose membrane is unbacked material with a capillary speed of 110-165 s/40 mm. The concentration of 15F11 for printing is between 0.5 mg/mL and 3 mg/mL. The total amount of antibody is between 0.2 μg antibody/cm and 5 μg antibody/cm of nitrocellulose. In one embodiment, the printing buffer is 20 mM Tris pH 8.0, although other buffers with other pH ranges (e.g., pH 6 through pH 9) may be used.

For embodiments with a latex particle as the reporter, the following procedure is used. The latex conjugate is diluted in a base buffer adjusted to a pH of 7 to 9. In one embodiment, the pH is 8.0 to 8.2 using either Tris or Borate. Carrier proteins are added to the base buffer. Immunoglobulins, fish skin gelatin, gelatin, albumin, and caseins may be used, and in one embodiment bovine serum albumin is utilized. Various sugars may be used to help with stability and rehydration of dried latex microspheres including the disaccharides trehalose and sucrose and the monosaccharides mannitol and sorbitol, at concentrations of 1% to 5%. In one embodiment, the sugar additive is trehalose used at 1% to 5%. Ionic and nonionic surfactants may be used to help with rehydration of dried latex microspheres. The concentration range is between 0.01% and 2%. In one embodiment, the surfactant is Tween-20.

Various polymers may be used to help with rehydration and mobilization of dried latex microspheres. The concentration range may be between 0.01% and 2%. Examples include polyvinylpyrrolidone, polyvinyl alcohol, and dextran. In one embodiment, polyvinylpyrrolidone (40,000 MW) at 0.5% is utilized. A range of materials may be used to store the dried latex microspheres including cellulose paper, glass fiber, and synthetic materials such as rayon. Blends of these materials may be used. In one embodiment, the material is a chopped glass fiber material.

False positives due to high IgM titer may be mitigated by the use of anti-human IgM antibodies added to the conjugate buffer. The anti IgM antibodies interfere with IgM activity. The anti IgM antibodies may be monoclonal (e.g. mouse, rat, rabbit), or polyclonal (e.g. goat, rabbit, sheep) at concentrations ranging from 0.1 to 5 mg/mL. Commercial products may be used in place of, or as a supplement to, anti IgM antibodies. Manufacturers of these additives include Meridian Diagnostics, Scantibodies, and Omega Biologicals. Additionally, the detector antibody may be proteolytically processed into F(ab′)₂ or F(ab) prior to coupling to microparticles to mitigate nonspecific binding of blood components.

The end pad material is contemplated as follows. An absorbent material is used to collect fluid after it passes through the nitrocellulose membrane. The material and dimensions are such that the fluid flows in only one direction for the duration of the test. Cellulose/cotton linter materials (Ahlstrom 270, Ahlstrom 238, or Whatman GB003), compressed cellulose, or similar are suitable end pads. In one embodiment, the wick material is Whatman GB003, Advantec 526 or Ahlstrom 222 chromatography paper.

In one embodiment, the sample pad is contemplated as follows. A sample pad to receive the blood sample is used to mitigate false positive results and to act as a filter for erythrocytes ensuring that only plasma or dilute plasma activates the dry chemistry of the conjugate pad. The sample pad may be a binder-free glass fiber pad with a mesh with a particle retention size of 1 micron to 4 microns. The sample pad may also be a combination of glass fiber and a synthetic material. The sample pad receives a treatment (sample pad buffer) to increase red cell retention and prevent false positives. The latex conjugate is diluted in a base buffer adjusted to a pH of 7 to 9. In one embodiment, the pH is 8.0 to 8.2 using either Tris or Borate. Carrier proteins are added to the base buffer. Immuoglobulins, fish skin gelatin, gelatin, albumin, and caseins may be used, but bovine serum albumin between 0.1% and 5% is used in one embodiment. Ionic and nonionic surfactants may be used to help with false positives and false negatives. The concentration range may be between 0.01% and 1%. In one embodiment, the surfactants are Tween-20 at 0.5% and polybrene at 0.5%.

Various polymers may be added to the base buffer. The concentration range may be between 0.01% and 5%. Examples include polyvinylpyrrolidone, polyvinyl alcohol, and dextran. In one embodiment, polyvinylpyrrolidone (40,000 MW) at 0.5% is utilized.

A wash reagent may be used to help with false positives and to ensure adequate sample volume to activate the dry chemistry. The wash reagent consists of a base buffer comprised of Tris or borate adjusted to a pH of 7 to 9. In one embodiment, the pH is in a range of 8.0 to 8.2. Sodium chloride (0.1M to 1M) may be added to the base buffer to increase stringency. In one embodiment, the sodium chloride concentration range is 0.15 to 0.3M. The wash reagent may be added after sample is directly added to the sample pad. Alternatively, the sample may be added to container with wash buffer, mixed, and then added to the sample pad to provide similar benefits for reducing background, and providing adequate volume to run the test.

In order to fully enable point-of-care and home testing, the test strip may be paired with a compact optoelectronic reader. One such optoelectronic reader contemplated is based on the prior art previously disclosed by the assignee in U.S. patent application Ser. No. 14/997,749, which is incorporated herein by reference, with certain modifications as noted in various embodiments described herein. One or more of the light sources is a low-cost UV source material, such as LEDs with peak wavelength between 350-400 nm. In one embodiment, reflectors are used to direct light and are made of a UV reflective material with >80% reflectivity from 350-400 nm. One such material is an aluminum film coating. Similarly, any materials in the optical path such as lenses or windows must be minimally (<20%) absorbing in the UV region from 350-400 nm. A photodectector with organic pigment filter for color selection or a photodetector with Gaussian filters integrated onto the chip provide optical sensing with wavelength selectivity. Optionally, an additional optical filter, such as a glass filter or gel filter, can be added to the optical path to selectively transmit light in the emission peak of the fluorescent particle. For Eu-chelate particles, a red filter with a transmittance over 90% at over 610 nm and <10% below 550 nm can be used. Either a cover or a test strip holder can be used to minimize stray light from entering the optical sensor by fully covering the test zone of the test strip when inserted into the optoelectronic reader. Using the architecture of the reader disclosed in U.S. patent application Ser. No. 14/997,749, an embodiment of a device in accordance with the present disclosure connects to a mobile device such as a phone or tablet through a headphone jack and alternatively can connect via Bluetooth® or other wireless protocols. Connectivity to a mobile device enables analysis to be done on the mobile device.

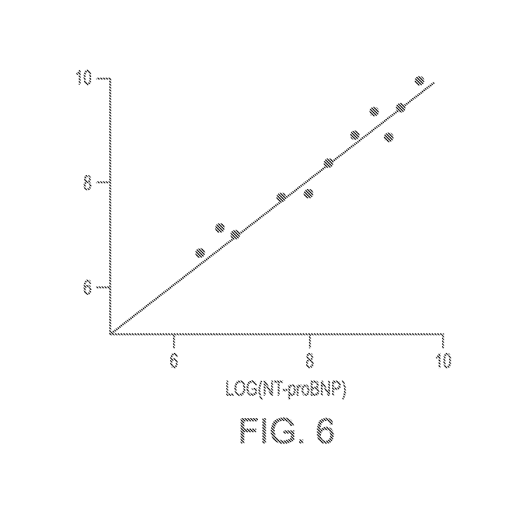

When the test strip is inserted into the optoelectronic reader and sample is added, the test runs and a signal is produced. The signal is analyzed using various methods to provide quantitative information about the concentration of NT-proBNP in the solution that was added. FIG. 6 is an example of data generated from one embodiment of the invention described herein.

Methods for analyzing the signal include the use of measuring the blank strip with or without a printed reference line as mentioned above as well as measuring the test zone at multiple time-points and with multiple wavelengths over the course of the test.

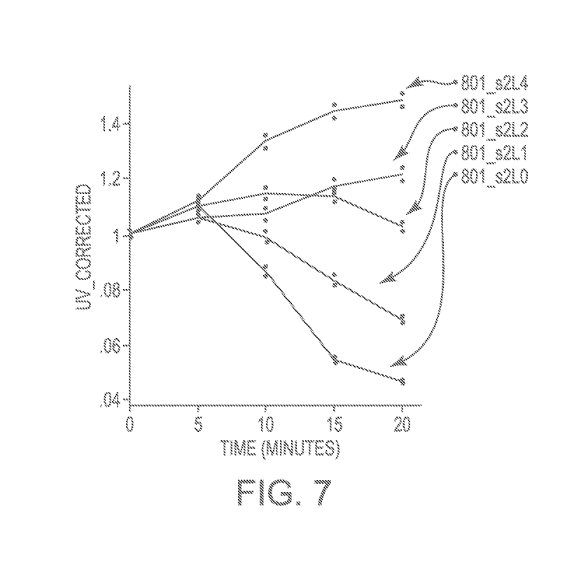

By reading the test at an initial time point and at specific intervals during development of the test result, the dynamic behavior of the test can be used to distinguish differences between samples of high concentration that would otherwise be difficult to differentiate by an end-point measurement due to the hook effect. FIG. 7 provides an example of one embodiment of this process wherein a test strip is read at 5 minute intervals for duplicates of samples with 5 different levels of NT-proBNP. More specifically, the trigger for the dynamic readout (ie. stopping criterion) could be either based on the rate of change or a threshold on one of the signals, or a feature that can combine both reflectance and fluorescence measurements. Potential such features that could be used are the intensity of the fluorescent test line that is measured (Fₜₑₛₜ), the intensity of the signal measured on the blank strip (F_{blank}) under UV excitation, the intensity of the reflected light from visible light sources while the test is running (Rₜₑₛₜ), the intensity of the reflected light from visible light sources on the blank strip (R_{blank}). Features that are combinations of the features listed above, such as F_{dfff}=Fₜₑₛₜ−F_{blank}, % F=Fₜₑₛₜ F_{blank}, % R=Rₜₑₛₜ/R_{blank}, F_{diff}/% R, % F/% R, KS=(1−% R)̂2*% R), F_{diff}/KS, % F/KS, may also be used. Non-limiting examples of the calibration curve that maps the feature to the NT-proBNP concentration include a polynomial of first or second degree, a 4-parameter logistic (4-PL) equation or a piece-wise linear look up table. It is further contemplated that software can be used to pick an appropriate calibration curve for the range of the sample based on the kinetics of the measured parameters.

Using a combination of reflectance photometry and fluorescence (or two fluorescent tags with different excitation wavelength or emission wavelengths, or two colored beads with different absorption wavelengths), the optoelectronic reader can simultaneously read the test and control line as they develop and use the dynamic formation to distinguish high levels. In embodiments where a fluorescent tag is used for one analyte and a visible tag for the other analyte, the choice of different detection modes enables low signal interference when reading the test through this method.

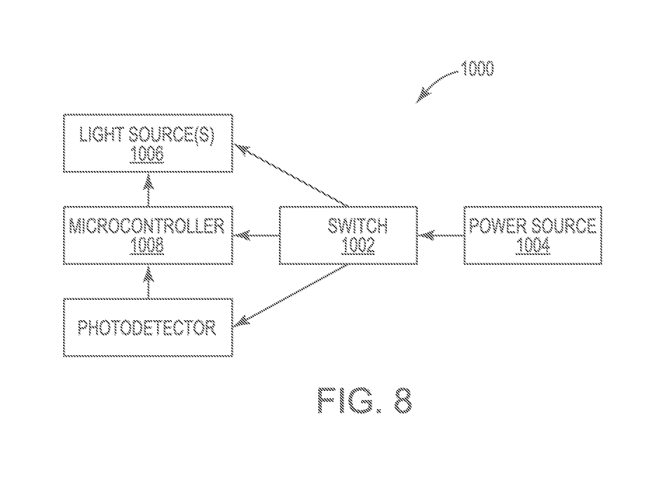

Referring now to FIG. 8, an embodiment of the architecture 1000 of the optoelectronic reader is depicted. A switch 1002 connects a power source 1004 to one or more light sources 1006, a microcontroller 1008, and a photodetector 1010. The switch 1002 is triggered by the insertion of the test strip into the optoelectronic reader and enables measurements to be taken. The microcontroller 1008 drives one or more light sources 1006 and the photodetector 1010. The microcontroller 1008 also processes the signal detected by the photodetector 1010 and communicates with the connected mobile device (not shown).

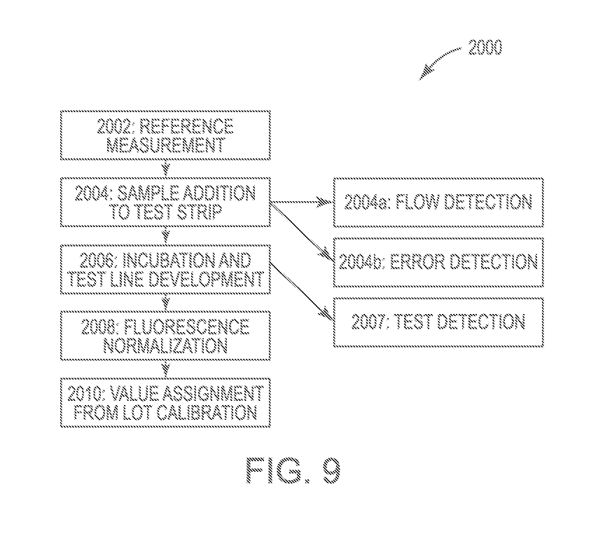

Referring now to FIG. 9, an embodiment of the test process 2000 for measuring the test strip with the optoelectronic reader is depicted. Upon insertion of the test strip, a reference measurement 2002 is made. When a sample is added to the test strip at 2004, a series of measurements are taken to detect flow of liquid in the test strip 2004a as well as to detect errors 2004b, such as the removal of the test strip. During an incubation time period at 2006, a test line develops on the test strip and can be read one or more times by the optoelectronic reader as part of the test detection at 2007. Measurements from the test detection are normalized at 2008 using the reference measurement and an NT-proBNP value is assigned at 2010 by applying a calibration curve specific to the lot of the test strip to the normalized signal.

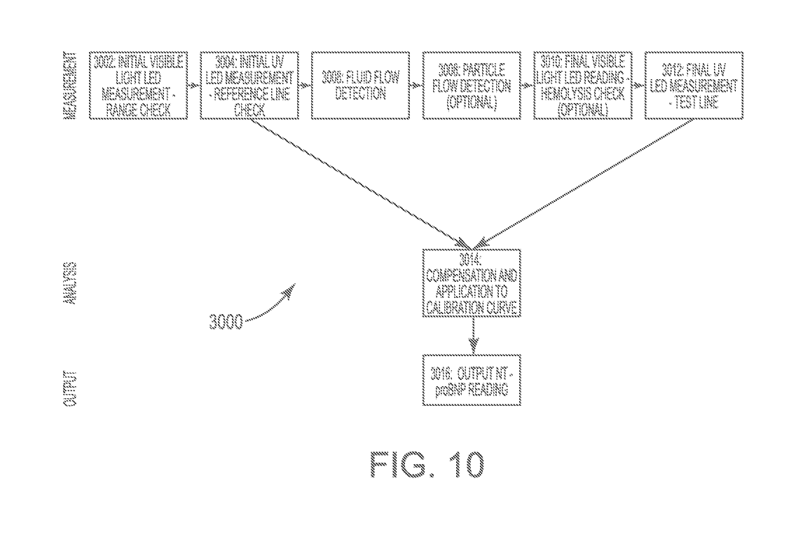

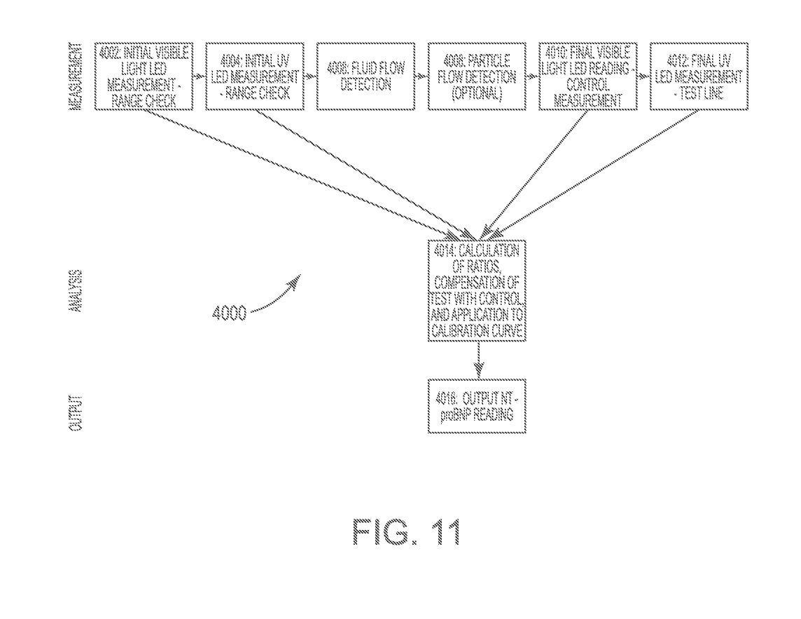

Referring now to FIG. 10 and FIG. 11, two non-limiting embodiments of normalization schemes 3000 and 4000, respectively, are depicted. In both schemes, an initial measurement is performed at 3002 and 4002, respectively, with a visible light LED, such as a red LED, to measure the strip before any sample has been added. This measurement can be used to perform a range check to disqualify a strip that has already been used, or that has been stained. An initial measurement using a UV LED is taken at 3004 and 4004, respectively, to measure a reference line specific to the lot of the test strip. Subsequently, measurements using the visible light LED are performed at 3006 and 4006, respectively, while sample is applied and are used to detect the flow of liquid onto the test strip. Optionally, at 3008 and 4008, respectively, the UV LED may be used to measure the flow of particles.

In the embodiment depicted in FIG. 10, an optional measurement at 3010 with the visible light LED after a set time may be used to detect hemolysis. The UV LED is then used to measure the final test line at 3012, and, at 3014, the value is compensated with the reference line measurement and applied to a calibration curve to assign an NT-proBNP value. The NT-proBNP value is then output to the user at 3016 through the interface on the mobile device that is connected to the optoelectronic reader.

In the embodiment depicted in FIG. 11, a measurement at 4010 with the red LED is used to measure the control line and then the UV LED is used to measure the test line at 4012. At 4014, these signals are compensated by the initial measurements with both the red and UV LED and ratios of the compensated values are applied to a calibration curve to assign an NT-proBNP value. The NT-proBNP value is then output to the user at 4016 through the interface on the mobile device that is connected to the optoelectronic reader.

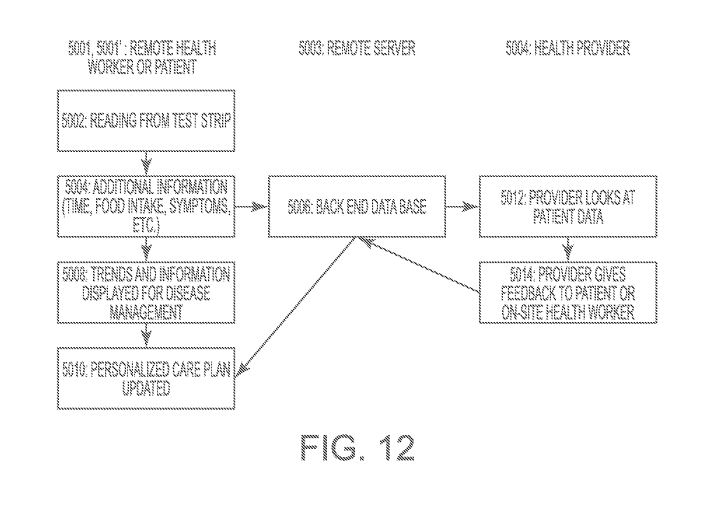

Referring now to FIG. 12, an embodiment of an application 5000 of the present invention is depicted wherein a remote health worker with a patient 5001 or the patient 5001′ themself performs testing that then communicates with a remote server 5003 and a health provider 5005 to provide real time feedback 5003 between the health provider and the patient. The reading 5002 from the test strip can be combined with other data 5004 gathered on a mobile application including time, location, food intake, symptoms, and weight, and then communicated to a database 5006. Trends and information 5008 can be displayed to assist with disease management. The data can also be used or reviewed 5012 by the provider to provide a personalized care plan 5014 to the patient.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A method of measuring the concentration of NT-proBNP in a sample of blood, serum or plasma comprising:
providing a lateral flow immunoassay test strip with a monoclonal antibody, 15F11 (a.a.r 13-24) bound to the solid phase, and monoclonal antibody, 24E11 (a.a.r. 67-76) or 16E6 (a.a.r. 34-39), conjugated fluorescent microparticles; and
using an optoelectronic reader that operably connects to a mobile device to:
obtain measurements of a fluorescent signal from the test strip after exposure to the sample; and
combine the measurements over time to provide an estimate of a concentration of NT-proBNP in the blood, serum, or plasma.

2. The method of claim 1 wherein providing the lateral flow immunoassay test strip comprises providing the test strip in which the fluorescent microparticles are polystyrene beads with Europium chelates.

3. The method of claim 2 wherein providing the lateral flow immunoassay test strip includes printing a reference line of a Europium chelate on the test strip.

4. The method of claim 3 wherein obtain measurements including generating a test signal that includes an assay of the reference line before the sample is added that is used to provide the estimate of the concentration of NT-proBNP.

5. The method of claim 3 wherein a test strip holder is used to align the reference line with the optoelectronic reader and allow introduction of the sample to the test strip.

6. The method of claim 2 where the polystyrene beads are covalently coupled to the monoclonal antibody, 24E11.

7. The method of claim 1 wherein the optoelectronic reader uses a light emitting diode with an emission peak between 350-400 nm to illuminate the test strip to obtain a test signal.

8. The method of claim 7 wherein the test signal is measured at multiple time points and wherein different time points are used to provide estimates of different levels of the concentration in the sample.

9. The method of claim 1 wherein the test strip is configured such that the sample is added to a side of the test strip that is opposite a side of the test strip from which a test signal is measured.

10. The method of claim 1 wherein a backing material for the test strip is a black opaque plastic.

11. The method of claim 1 wherein a backing material for the test strip is a clear plastic material with a transmittance between 350-400 nm of more than 80%.

12. The method of claim 1 wherein the optoelectronic reader is connected to a mobile device via an audio jack.

13. The method of claim 1 wherein the optoelectronic reader is connected to a mobile device via a wireless connection.

14. The method of claim 1 wherein the sample is added to the test strip and subsequently a wash buffer is added to the test strip to reduce background signal.

15. The method of claim 1 wherein the sample is added to a dilution reagent, mixed, and then added to the test strip.

16. A system to test for analyte NT-proBNP in whole blood, serum, or plasma using an optoelectronic reader accessory to analyze test strips with a mobile device, the mobile device including a central processing unit, the system comprising:
a test strip including—
a sample pad configured to retain blood cells while allowing fluid to pass through passive wicking on the test strip,
a conjugate pad configured to release fluorescently labeled antibodies upon contact with fluid wicking on the test strip, and
a membrane sensitized with a test line configured to selectively capture NT-proBNP, the membrane being in fluid connection with said conjugate pad on the test strip; and
an optoelectronic reader accessory configured to be coupled to the mobile device, the accessory including—
structure defining an aperture configured to permit light to pass into and out of the optoelectronic reader at a defined viewing axis,
structure defining a slot for operable insertion of the test strip, the slot being configured to orient the test strip to intersect the defined viewing axis,
a microcontroller configured to control electronic components on a circuit within the optoelectronic reader and to operably communicate with the central processing unit of the mobile device,
at least one light source configured to emit light at a wavelength between 300-400 nm,
a reflective surface configured to direct light from the at least one light source to a surface of a test zone of the test strip,
an optical system configured to detect optical signals from the test strip, and
a power source disposed within the optoelectronic reader and operatively coupled with the microcontroller, at least one light source, and the optical system,
wherein the accessory is configured to communicate signals representative of optical signals from the optical system to the central processing unit of the mobile device to be analyzed to provide an estimate of a concentration of NT-proBNP in the blood, serum, or plasma.

17. The system of claim 16, wherein a reference fluorescent material is co-located on the test line of the test strip.

18. The system of claim 16, wherein the test strip is housed within a cassette that fits into the optoelectronic reader accessory.

19. The system of claim 16, further comprising a circuit in the optoelectronic reader configured to detect an out-of-range condition.

20. The system of claim 19, wherein the circuit is one of a low battery detection circuit and a temperature out-of-range circuit.

21. The system of claim 16, wherein the reference fluorescent material comprises a europium chelate.

22. The system of claim 16, wherein an optical path of the optoelectronic reader includes a filter after emission of light from the test zone.

23. The system of claim 16, wherein the test strip further comprises an end pad configured to provide wicking flow throughout the test strip, the end pad being in fluid connection with the membrane on the test strip.

24. A method of measuring the concentration of an analyte in a fluid, the method comprising:
providing a lateral flow immunoassay test strip configured to release fluorescently or visually labeled markers;
providing an optoelectronic reader couplable to a mobile device to analyze signals obtained from the test strip;
providing instructions for using the lateral flow immunoassay test strip in combination with the optoelectronic reader and the mobile device, the instructions including the steps of:
depositing a sample of the fluid on the test strip,
inserting the test strip into the optoelectronic reader for analysis to obtain measurements of a fluorescent signal from the test strip after exposure to the sample, and
using the mobile device to obtain an estimate of a concentration of analyte in the sample based on measurement over time of the test strip.
